(19) United States
(12) Patent Application Publication
(10) Pub. No.: US 2007/0275472 A1
(43) Pub. Date: Nov. 29, 2007

(54) METHOD FOR DETECTING PRESENCE OF SILVER-CONTAINING ANTIMICROBIAL AGENTS

(75) Inventor: Sidney J. Bertucci, Rochester, NY (US)

Correspondence Address: Paul A. Leipold, Patent Legal Staff;Eastman Kodak Company, 343 State Street, Rochester, NY, 14650-2201, US

(73) Assignee: Eastman Kodak Company

(21) Appl. No.: 11/419,927

(22) Filed: May 23, 2006

(51) Int. Cl. G01N 33/20

(52) U.S. Cl. 436/80

(57) ABSTRACT

A method for testing for presence of silver metal or silver salt antimicrobial agents on a surface of substrate, comprising a) contacting the substrate with a dye solution, wherein the dye solution comprises a dye selected to provide a detectable differential color change in the dye solution contacted substrate for a substrate having silver metal or silver salt on a surface thereof relative to a substrate not having silver metal or silver salt on a surface thereof, and b) detecting a presence or absence of the differential color change in the dye solution contacted substrate.

### FIELD OF THE INVENTION

The present invention relates to a method for detecting presence of silver-containing antimicrobial agents on a surface of a substrate or in treatment solutions. More particularly, it relates to testing for presence of antimicrobial silver metal and silver salts that may be applied to a substrate such as a target fiber or textile fabric.

### BACKGROUND OF THE INVENTION

The antimicrobial properties of silver have been known for several thousand years. The general pharmacological properties of silver are summarized by Stewart C. Harvey in Chapter 41, “Antiseptics and Disinfectants: Fungicides; Ectoparasiticides” (pp 964-987) of The Pharmacological Basis of Therapeutics, Fifth Edition, by Louis S. Goodman and Alfred Gilman (editors), published by MacMillan Publishing Company, NY, 1975 (see “Heavy Metals and Their Salts”, pp 975-977). It is now understood that the affinity of silver ion for biologically important moieties such as sulfhydryl, amino, imidazole, carboxyl and phosphate groups are primarily responsible for its antimicrobial activity.

The attachment of silver ions to one of these reactive groups on a protein results in the precipitation and denaturation of the protein. The extent of the reaction is related to the concentration of silver ions. The diffusion of silver ion into mammalian tissues is self-regulated by its intrinsic preference for binding to proteins through the various biologically important moieties on the proteins, as well as precipitation by the chloride ions in the environment. Thus, the very affinity of silver ion to a large number of biologically important chemical moieties (an affinity which is responsible for its action as a germicidal/biocidal/viricidal/fungicidal/bacteriocidal agent) is also responsible for limiting its systemic action—silver is not easily absorbed by the body. This is a primary reason for the tremendous interest in the use of silver containing species as an antimicrobial i.e. an agent capable of destroying or inhibiting the growth of microorganisms, including bacteria, yeast, fungi and algae, as well as viruses.

In addition to the affinity of silver ions for biologically relevant species, which leads to the denaturation and precipitation of proteins, some silver compounds, those having low ionization or dissolution ability, also function effectively as antiseptics. Distilled water in contact with metallic silver bercomes antibacterial even though the dissolved concentration of silver ions is less than 100 ppb. There are numerous mechanistic pathways by which this oligodynamic effect is manifested i.e. by which silver ion interferes with the basic metabolic activities of bacteria at the cellular level, thus leading to a bacteriocidal and/or bacteriostatic effect.

A detailed review of the oligodynamic effect of silver can be found in “Oligodynamic Metals” by I. B. Romans in Disinfection, Sterlization and Preservation, C. A. Lawrence and S. S. Bloek (editors), published by Lea and Fibiger (1968) and “The Oligodynamic Effect of Silver” by A. Goetz, R. L. Tracy and F. S. Harris, Jr. in Silver in Industry, Lawrence Addicks (editor), published by Reinhold Publishing Corporation, 1940. These reviews describe results that demonstrate that silver is effective as an antimicrobial agent towards a wide range of bacteria.

One very important use of silver based antimicrobial agents is for textiles. Various methods are known in the art to render antimicrobial properties to a target fiber. The approach of embedding inorganic antimicrobial agents, such as zeolites, into low melting components of a conjugated fiber is described in U.S. Pat. No. 4,525,410 and U.S. Pat. No. 5,064,599. In another approach, the antimicrobial agent may be delivered during the process of making a synthetic fiber such as those described in U.S. Pat. No. 5,180,402, U.S. Pat. No. 5,880,044, and U.S. Pat. No. 5,888,526, or via a melt extrusion process as described in U.S. Pat. No. 6,479,144 and U.S. Pat. No. 6,585,843. In still yet another process an antimicrobial metal ion may be ion exchanged with an ion exchange fiber as described in U.S. Pat. No. 5,496,860.

Methods of transferring an antimicrobial agent, in the form of an inorganic metal salt or zeolite, from one substrate to a fabric are disclosed in U.S. Pat. No. 6,461,386. High-pressure laminates containing antimocrobial inorganic metal compounds are disclosed in U.S. Pat. No. 6,248,342. Deposition of antimicrobial metals or metal-containing compounds onto a resin film or target fiber has also been described in U.S. Pat. No. 6,274,519 and U.S. Pat. No. 6,436,420.

It is also known in the art that fibers may be rendered with antimicrobial properties by applying a coating of silver containing antimicrobial compositions. Silver ion-exchange compounds, silver zeolites and silver glasses are all known to be applied to fibers through topical applications for the purpose of providing antimicrobial properties to the fiber as described in U.S. Pat. No. 6,499,320, U.S. Pat. No. 6,584,668, U.S. Pat. No. 6,640,371 and U.S. Pat. No. 6,641,829. Other inorganic antimicrobial agents may be contained in a coating that is applied to a fiber as described in U.S. Pat. No. 5,709,870, U.S. Pat. No. 6,296,863, U.S. Pat. No. 6,585,767 and U.S. Pat. No. 6,602,811. It is known in the art to use binders to apply coating compositions to impart antimicrobial properties to various substrates. U.S. Pat. No. 6,716,895 describes the use of hydrophilic and hydrophobic polymers and a mixture of oligodynamic metal salts as an antimicrobial composition, wherein the water content in the coating composition is preferably less than 50%. U.S. Pat. No. 5,709,870 describes the use of carboxymethyl cellulose-silver complexes to provide an antimicrobial coating to a fiber. The use of silver halides in an antimicrobial coating, particularly for medical devices, is described in U.S. Pat. No. 5,848,995.

US 2006/0068024 describes aqueous compositions for coating a fabric or fiber with an antimicrobial comprising water, silver halide particles and a hydrophilic polymer. It further describes a composition comprising at least two separately packaged parts, the first part being a composition comprising water, silver halide particles and a hydrophilic polymer; and the second part being a composition comprising an aqueous suspension of a hydrophobic binder, or a composition comprising a crosslinker for the hydrophilic polymer. The preferred hydrophilic polymer is gelatin. Also described is a method of coating a fabric or fiber comprising mixing the two separately packaged parts; and coating the mixture on fabric or fiber. The described compositions impart durable antimicrobial properties to yarn, fabrics or textiles. The silver halide particles are applied to the target fiber or textile fabric with the aid of a hydrophilic binder that imparts colloidal stability to the particles prior to and during the application process to the fiber or textile fabric. The composition may also be aided with the use of a hydrophobic binder to impart improved durability to extended washing cycles that would otherwise remove the particles and the associated antimicrobial properties of the fiber or textile fabric.

Various approaches to and compositions for treating substrates such as fibers and textile fabrics with silver antimicrobial compositions have been proposed. It is generally desired, however, that the applied antimicrobial composition does not negatively affect the other properties of the treated substrate. In particular, the antimicrobial compositions are typically desirably applied in an appropriate manner and at a concentration so as not to significantly change the visual appearance of the substrate. This desirable feature, however, makes it difficult to easily verify whether an antimicrobial agent is actually present in a treatment solution, and has actually been effectively applied to a substrate. It accordingly would be advantageous to provide a method for detecting the presence of silver-containing antimicrobial agents on the surface of a substrate, or presence of such agents in a treatment solution, to verify whether a substrate has actually been treated with a silver antimicrobial composition.

### SUMMARY OF THE INVENTION

In accordance with one embodiment, the present invention is directed towards a method for testing for presence of silver metal or a silver salt on a surface of substrate, comprising a) contacting the substrate with a dye solution, wherein the dye solution comprises a dye selected to provide a detectable differential color change in the dye solution contacted substrate for a substrate having silver metal or silver salt on a surface thereof relative to a substrate not having silver metal or silver salt on a surface thereof, and b) detecting a presence or absence of the differential color change in the dye solution contacted substrate.

In accordance with a further embodiment, the invention is also directed towards a method of coating a substrate with a silver-containing composition to provide antimicrobial properties comprising: providing a composition comprising a silver-containing antimicrobial agent; providing a substrate; coating the substrate with said composition; and verifying presence of silver-containing antimicrobial agent deposited on a surface of the substrate by contacting the substrate with a dye solution, wherein the dye solution comprises a dye selected to provide a detectable differential color change in the dye solution contacted substrate for a substrate having silver-containing antimicrobial agent on a surface thereof relative to a substrate not having silver-containing antimicrobial agent on a surface thereof, and detecting a presence or absence of the differential color change in the dye solution contacted substrate.

In accordance with a further embodiment, the invention is also directed towards a method for testing for presence of silver-containing antimicrobial agent in a treatment solution, comprising a) contacting a sample of the treatment solution with a dye solution, wherein the dye solution comprises a dye selected to provide a detectable differential color change in the dye solution contacted treatment solution for a treatment solution having silver-containing antimicrobial agent therein relative to a treatment solution not having silver-containing antimicrobial agent therein, and b) detecting a presence or absence of the differential color change in the dye solution contacted treatment solution. In such embodiment, the treatment solution may be subsequently used to coat a substrate with the silver-containing antimicrobial agent.

### BRIEF DESCRIPTION OF THE DRAWINGS

FIG. 1 is a graph showing the reflection optical density of the dyed samples of Examples 2a-2d measured as a function of wavelength.

FIG. 2 is a graph showing the Delta Reflection Densities in Table 2 of Example 2 plotted as a function of padded silver chloride level.

### DETAILED DESCRIPTION OF THE INVENTION

The present invention in one embodiment provides a method for testing for presence of silver metal or a silver salt deposited on a surface of a substrate. In such method, the substrate to be tested is contacted with a dye solution, wherein the dye solution comprises a dye selected to provide a detectable differential color change in the dye solution contacted substrate for a substrate having silver metal or silver salt on a surface thereof relative to a substrate not having silver metal or silver salt on a surface thereof. In various particular embodiments, the dye solution may comprise a dye selected to have adsorption affinity for a silver salt, and the dye solution may test directly for the presence of a silver salt, or test indirectly for the presence of silver metal by converting a surface of the silver metal in situ to a silver salt. Upon adsorption of the dye, a detectable differential color change in the dye solution contacted substrate is obtained for a substrate having silver metal or silver salt on a surface thereof relative to a substrate not having silver metal or silver salt on a surface thereof. By detecting a presence or absence of the differential color change in the dye solution contacted substrate, the presence or absence of silver metal or silver salt may be confirmed.

In a particular embodiment the invention is directed specifically towards a test for the presence of silver halide particles. As taught in US 2006/0068024 referenced above, e.g., the disclosure of which is incorporated herein by reference in its entirety, silver halide dispersions have been found to be effective for treating substrates such as fibers and textile fabrics to provide antimicrobial properties. Photographic sensitizing dyes are a well known class of dyes known to have adsorption affinity for silver halide particles, and may be conveniently employed as the dye in the dye solution in such embodiment.

It is common in the art of spectral sensitization of silver halide emulsions, e.g., to use cyanine dyes that transfer the energy of adsorbed light to the conduction band of the silver halide, thus making the silver halide sensitive to wavelengths longer than its native sensitivity. Along with the ability to transfer the energy of adsorbed light to the silver halide, sensitizing dyes must also have the ability to effectively adsorb to silver halide so as to enable such transfer of energy. It is this known affinity for adsorption to silver halide that make such known class of dyes appropriate for use in the present invention. Photographic sensitizing dyes are well known in the art and are disclosed, for example, in Research Disclosure, September 1996, 38957, Section V. Research Disclosure is published by Kenneth Mason Publications, Ltd., Dudley House, 12 North St., Emsworth, Hampshire PO10 7DQ, England. The dyes useful in the invention can be prepared by synthetic techniques well known in the art. Such techniques are further illustrated, for example, in “The Cyanine Dyes and Related Compounds”, Frances Hamer, Interscience Publishers, 1964 and “The Theory of the Photographic Process”, T. H. James, ed., 4th Edition, Macmillan (1977).

The dye solutions used in the invention can employ dyes from a variety of classes, including the polymethine dye class, which includes the cyanines, merocyanines, complex cyanines and merocyanines (i.e., tri-, tetra- and polynuclear cyanines and merocyanines), styryls, merostyryls, streptocyanines, hemicyanines, arylidenes, allopolar cyanines and enamine cyanines. Cyanine dyes include, joined by a methine linkage, two basic heterocyclic nuclei, such as those derived from quinolinium, pyridinium, isoquinolinium, 3H-indolium, benzindolium, oxazolium, thiazolium, selenazolinium, imidazolium, benzoxazolium, benzothiazolium, benzoselenazolium, benzotellurazolium, benzimidazolium, naphthoxazolium, naphthothiazolium, naphthoselenazolium, naphtotellurazolium, thiazolinium, dihydronaphthothiazolium, pyrylium and imidazopyrazinium quaternary salts. Merocyanine dyes include, joined by a methine linkage, a basic heterocyclic nucleus of the cyanine-dye type and an acidic nucleus such as can be derived from barbituric acid, 2-thiobarbituric acid, rhodanine, hydantoin, 2-thiohydantoin, 4-thiohydantoin, 2-pyrazolin-5-one, 2-isoxazolin-5-one, indan-1,3-dione, cyclohexan-1,3-dione, 1,3-dioxane-4,6-dione, pyrazolin-3,5-dione, pentan-2,4-dione, alkylsulfonyl acetonitrile, benzoylacetonitrile, malononitrile, malonamide, isoquinolin-4-one, chroman-2,4-dione, 5H-furan-2-one, SH-3-pyrrolin-2-one, 1,1,3-tricyanopropene and telluracyclohexanedione.

Among useful spectral sensitizing dyes known to have adsorption affinity for silver halide are those found in U.K. Patent 742,112, Brooker U.S. Pat. Nos. 1,846,300, '301, '302, '303, '304, 2,078,233 and 2,089,729, Brooker et al U.S. Pat. Nos. 2,165,338, 2,213,238, 2,493,747, '748, 2,526,632, 2,739,964 (Reissue 24,292), U.S. Pat. No. 2,778,823, 2,917,516, 3,352,857, 3,411,916 and 3,431,111, Sprague U.S. Pat. No. 2,503,776, Nys et al U.S. Pat. No. 3,282,933, Riester U.S. Pat. No. 3,660,102, Kampfer et al U.S. Pat. No. 3,660,103, Taber et al U.S. Pat. Nos. 3,335,010, 3,352,680 and 3,384,486, Lincoln et al U.S. Pat. No. 3,397,981, Fumia et al U.S. Pat. Nos. 3,482,978 and 3,623,881, Spence et al U.S. Pat. No. 3,718,470 and Mee U.S. Pat. No. 4,025,349, the disclosures of which are here incorporated by reference. One or more dyes may be employed in combination.

Furthermore, in the spectral sensitization of silver halide emulsions for color photographic applications, it is customary to use J-aggregating cyanine dyes because of the narrower light absorption of the aggregate and the improved color separation that it provides (see The Theory of the Photographic Process, 4th edition, T. H. James, editor, Macmillan Publishing Co., New York, 1977, for a discussion of aggregation). Use of J-aggregating cyanine dyes may be particularly advantageous for providing a signal for the presence of silver halide on a substrate in accordance with the method of the present invention.

The following illustrate specific dyes that may be employed in various embodiments of the present invention:

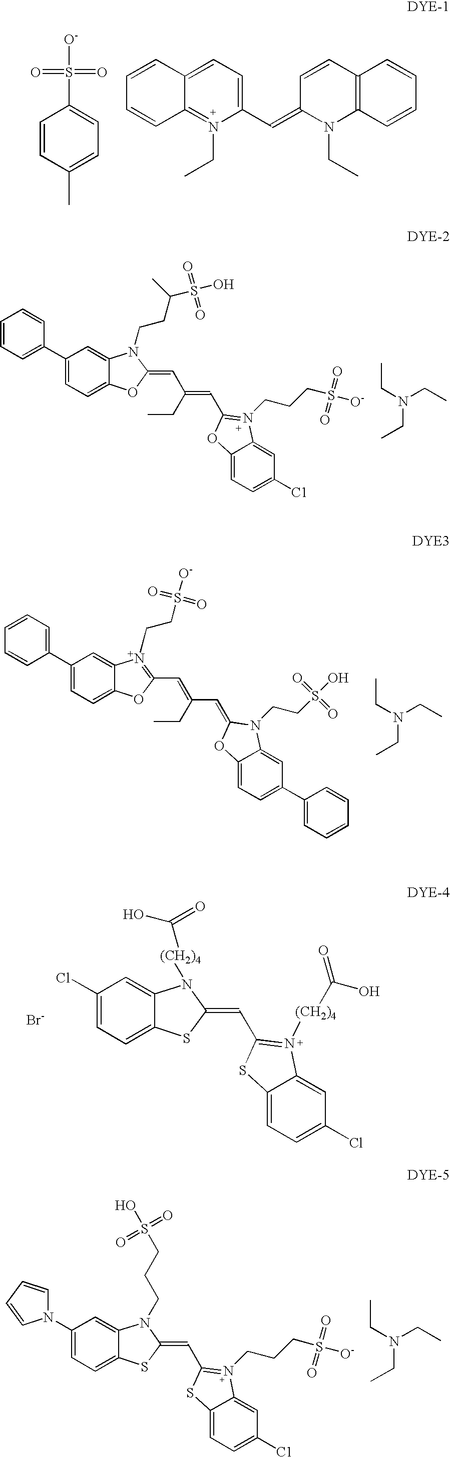

Dye solutions employed in the present invention are preferably aqueous based, but may also preferably comprise a minor percentage of organic solvent such as methanol. In a particularly preferred embodiment, dye solutions employed to test for the presence of silver halide salts on a substrate may include soluble halide salts, and in particular bromide or iodide salts, in an amount effective to enhance the detectable differential color change in the dye solution contacted substrate obtained for a substrate having silver halide particles on a surface thereof relative to a substrate not having silver halide particles on a surface thereof, as the presence of such halide ions in solution has been found to enhance the adsorption of sensitizing dyes on silver halide surfaces, especially when the silver halide is predominantly silver chloride. In an alternative embodiment, dye solutions containing soluble halide salts may be employed in the present invention to test for the presence of metallic silver on a substrate, wherein the soluble halide salt serves the purpose of facilitating conversion of the surface of the metallic silver to silver halide in the presence of environmental oxygen (or other added oxidizing agent) dissolved in the dye solution. In this embodiment, soluble iodide is particularly preferred for inclusion in the dye solution.

Dyes may be selected for use in the present invention such that adsorption of dye to silver halide particles on a substrate preferably results in a differential color change that is human visually detectable. Accordingly when employing a colored substrate, it may be preferable to select a dye from known colored dyes based on the color of the substrate to provide a human visually detectable differential color change. Even if not readily human visually detectable, dyes may be selectively adsorbed such that the presence or absence of silver on a substrate results in a differential optical reflection density spectrum for the dye solution contacted substrate.

The present invention further provides a method of coating a substrate with a silver-containing composition to provide antimicrobial properties comprising: providing a composition comprising a silver-containing antimicrobial agent; providing a substrate; coating the substrate with said composition; and verifying presence of silver-containing antimicrobial agent deposited on a surface of the substrate by contacting the substrate with a dye solution, wherein the dye solution comprises a dye selected to provide a detectable differential color change in the dye solution contacted substrate for a substrate having silver-containing antimicrobial agent on a surface thereof relative to a substrate not having silver-containing antimicrobial agent on a surface thereof, and detecting a presence or absence of the differential color change in the dye solution contacted substrate. The composition preferably comprises water, silver halide particles, and a binder; and the dye solution preferably comprises a dye having a greater adsorption affinity for silver halide relative to that for the substrate or has a detectable different color when adsorbed to silver halide relative to color when adsorbed to the substrate.

The method of the invention may be performed on an actual end product treated with silver-containing treatment solution to instill antimicrobial properties, or alternatively, the present invention may be employed on a test substrate previously treated with a substrate treatment solution to verify presence or absence of silver-containing antimicrobial agent in the substrate treatment solution, prior to use of the treatment solution to actually treat a desired end product. In such latter embodiment, it may be desirable to select a test substrate that provides a human visually detectable differential color change, especially where use of a dye solution for detection of silver on the actual desired end product may not provide a strong visual signal.

In a further embodiment, the invention provides a method for testing for presence of silver-containing antimicrobial agent in a treatment solution itself, such as a treatment solution intended for processing a substrate to coat the substrate with silver-containing antimicrobial agent. In such embodiment, a sample of the treatment solution is contacted with a dye solution, wherein the dye solution comprises a dye selected to provide a detectable differential color change in the dye solution contacted treatment solution for a treatment solution having silver-containing antimicrobial agent therein relative to a treatment solution not having silver-containing antimicrobial agent therein, and a presence or absence of the differential color change in the dye solution contacted treatment solution is detected to verify the presence or absence of silver-containing antimicrobial agent. The treatment solution may comprise, e.g., water, silver halide particles, and a binder; and the dye solution may comprise a dye having adsorption affinity for silver halide and which provides a detectable different color when adsorbed to silver halide relative to color when not adsorbed to silver halide. Similarly as when testing for the presence of silver metal on a surface of substrate, dye solutions containing soluble halide salts may be employed in the present invention to test for the presence of metallic silver in a treatment solution, wherein the soluble halide salt serves the purpose of facilitating conversion of the surface of the metallic silver to silver halide in the presence of environmental oxygen (or other added oxidizing agent) dissolved in the dye solution. Additionally, dye solutions containing soluble halide salts may be employed in the present invention to test for the presence of soluble silver in a treatment solution, wherein the soluble halide and soluble silver react in situ to precipitate silver halide particles to which the dye of the dye solution may adsorb.

As taught in US 2006/0068024, silver halide antimicrobial compositions preferably may comprise at least 50% water by weight, silver halide particles, and a hydrophilic polymer. The hydrophilic polymer preferably is of a type and used in an amount wherein the composition does not substantially gel or solidify at 25 degrees C. In practical terms the composition, when sold as a concentrate, should be able to flow at 25 degrees C. and be easily mixed with an aqueous diluent or other addenda prior to use as an antimicrobial coating for yarn or textile. The composition also encompasses a more diluted form that is suitable for dip, pad, or other types of coating. In one embodiment, e.g., it comprises at least 70% water by weight. In its most diluted form the composition may be greater than 95% water. The composition is preferably substantially free of organic solvents. Preferably, no organic solvent is intentionally added to the composition. The composition exhibits antimicrobial activity upon drying.

The silver halide particles may be of any shape and halide composition. The type of halide may include chloride, bromide, iodide and mixtures of them. The silver halide particles may be, for example, silver bromide, silver iodobromide, bromoiodide, silver iodide or silver chloride. In one embodiment the silver halide particles are predominantly silver chloride. The predominantly silver chloride particles may be, for example, silver chloride, silver bromochloride, silver iodochloride, silver bromoiodochloride and silver iodobromochloride particles. By predominantly silver chloride, it is meant that the particles are greater than about 50 mole percent silver chloride. Preferably, they are greater than about 90 mole percent silver chloride; and optimally greater than about 95 mole percent silver chloride. The silver halide particles may either be homogeneous in composition or the core region may have a different composition than the shell region of the particles. The shape of the silver halide particles may be cubic, octahedral, tabular or irregular. More silver halide properties may be found in “The Theory of the Photographic Process”, T. H. James, ed., 4th Edition, Macmillan (1977). In one embodiment the silver halide particles have a mean equivalent circular diameter of less than 1 micron, and preferably less 0.5 microns.

The solubility of silver halide, hence the free silver ion concentration, is determined by the solubility product (Ksp), particle size, structure and shape of the particle. While not being held to the theory, it is believed that the free silver ion concentration plays a role in antimicrobial efficacy. By controlling the above variables one can control silver ion release rate and antimicrobial activity.

The silver halide particles and associated coating composition preferably may be applied to a substrate such as a fiber or fabric in an amount sufficient to provide antimicrobial properties to the treated substrate for a minimum of at least 10 washes, more preferably 20 washes and most preferably after 30 washes in accordance with the standard domestic washing and drying procedure for textile testing ISO 6330:2003 published by the International Organization for Standardization, Geneva, Switzerland. The amount of silver halide particles applied to the target substrate is determined by the desired durability or length of time of antimicrobial properties. The amount of silver halide particles present in the composition will depend on whether the composition is one being sold in a concentrated form suitable for dilution prior to coating or whether the composition has already been diluted for coating. Typical levels of silver salt particles (by weight percent) in the formulation are preferably from about 0.000001% to about 10%, more preferably from about 0.0001% to about 1% and most preferably from about 0.001% to 0.5%. In a concentrated format the composition preferably comprises silver halide particles in an amount of 0.001 to 10%, more preferably 0.001 to 1%, and most preferably 0.001 to 0.5%. In a diluted format the composition preferably comprises silver halide particles in an amount from about 0.000001% to about 0.01%, more preferably from about 0.00001% to about 0.01% and most preferably from about 0.0001% to 0.01%. It is a desirable feature to provide efficient antimicrobial properties to the target substrate at a minimum silver halide level to minimize the cost associated with the antimicrobial treatment.

The preferred hydrophilic polymers employed in silver halide particle antimicrobial compositions coated in particular embodiments of the present invention are soluble in water at concentrations greater than about at least 2%, preferably greater than 5%, and more preferably greater than 10%. Therefore, suitable hydrophilic polymers do not require an organic solvent to remain fluid at 25 degrees C. Suitable useful hydrophilic polymers include, for example, gelatin, polyacrylic acid, polyacrylamide, polyvinyl alcohol, polyvinylpyrrolidones, cellulose etc. The polymers peptize or stabilize silver halide particles to help maintain colloidal stability of the solution. A preferred hydrophilic polymer is gelatin.

Gelatin is an amphoteric polyelectrolyte that has excellent affinity to a number of substrates. Gelatin may be processed by any of the well-known techniques in the art including; alkali-treatment, acid-treatment, acetylated gelatin, phthalated gelatin or enzyme digestion. The gelatin may have a wide range of molecular weights and may include low molecular weight gelatins if it is desirable to raise the concentration of the gelatin in the antimicrobial composition without solidifying the composition. The gelatin is preferably added in an amount sufficient to peptize the surface of the silver halide and some excess of gelatin will always be present in the water phase. The gelatin level may be chosen such that the composition does not substantially solidify or gel. In one embodiment the weight percentage of gelatin is less than 3%, preferably less than 2%, and more preferably less than 1%. The gelatin may also be cross-linked in order to improve the durability of the coating composition containing the antimicrobial silver halide particles.

Silver halide particles may be formed by reacting silver nitrate with halide in aqueous solution. In the process of silver halide precipitation one can add hydrophilic polymers to peptize the surface of the silver halide particles thereby imparting colloidal stability to the particles, see for example, Research Disclosure September 1997, Number 40122 published by Kenneth Mason Publications, Ltd., Dudley Annex, 12a North Street, Emsworth, Hampshire PO10 7DQ, ENGLAND, the contents of which are incorporated herein by reference.

In addition to hydrophilic binder, a hydrophobic binder resin is preferably used to improve the adhesion and durability of the silver salt particles once applied to the substrate surface. Such hydrophobic binders are well known in the art and are typically provided as aqueous suspensions of polymer microparticles. Materials suitable for use as hydrophobic binders include acrylic, styrene-butadiene, polyurethane, polyester, polyvinyl acetate, polyvinyl acetal, vinyl chloride and vinylidine chloride polymers, including copolymers thereof. Acrylic polymers and polyurethane are preferred.

The hydrophobic binders should have film-forming properties that include a range of glass transition temperatures from about −30 C to about 90 C. The hydrophobic binder particles may have a wide range of particle sizes from about 10 nm to about 10,000 nm and can be polydisperse in distribution. The hydrophobic binders may also be thermally or chemically crosslinkable in order to modify the desired durability properties of the antimicrobial composition treated substrate (e.g., fiber or fabric textile). The hydrophobic binders may be nonionic or anionic in nature. Useful ranges of the hydrophobic binders are generally less than about 10% of the composition. It is understood that the choice of the hydrophobic binder may be related to specific end use requirements of the substrate, including, e.g., wash resistance, abrasion (crock), tear resistance, light resistance, coloration, hand and the like for fiber or fabric textile substrates. As described in more detail below the hydrophobic binder is generally preferably kept separate from the hydrophilic polymer/silver halide particle composition until a short time prior to coating.

As noted above, the antimicrobial composition may also comprise a crosslinker for the gelatin. The crosslinker is also generally kept separate from the hydrophilic polymer/silver halide particle composition until a short time prior to coating. Examples of compounds useful in crosslinking the gelatin include, but are not limited to, Alum, formaldehyde and free dialdehydes such as glutaraldehyde, bis(iminomethyl)ether salts, strazines and diazines, such as dihydroxychlorotriazine, epoxides, aziridines, and the like.

Treatment solutions comprising a silver-containing antimicrobial agent, and in particular antimicrobial compositions comprising silver-containing antimicrobial agent, hydrophilic binder and optionally, hydrophobic binder or gelatin cross-linker, can be applied to a target substrate, such as a fiber or textile fabric, in any of the well know methods in art including, pad coating, knife coating, screen coating, spraying, foaming and kiss-coating. In a specific embodiment, components of the antimicrobial composition are preferably delivered as a separately packaged two-part system involving colloidal silver halide particles and hydrophilic binder as one part and a second part comprising an aqueous suspension of a hydrophobic binder, or gelatin cross-linker and, optionally, a second hydrophilic binder that may be the same or different as the hydrophilic binder from the first part. The first part, comprising colloidal silver halide particles and hydrophilic binder, is excellent in shelf-life without compromising colloidal stability. The two parts may be combined prior to a padding or coating operation and exhibit colloidal stability for the useful shelf-life of the composition, typically on the order of several days.

There may also be present further optional components, for example, thickeners or wetting agents to aid in the application of the antimicrobial composition to the target substrate. Examples of wetting materials include surface active agents commonly used in the art such as ethyleneoxide-propyleneoxide block copolymers, polyoxyethylene alkyl phenols, polyoxyethylene alkyl ethers, and the like. Compounds useful as thickeners include, for example, particulates such as silica gels and smectite clays, polysaccharides such as xanthan gum, polymeric materials such as acrylic-acrylicacid copolymers, hydrophobically modified ethoxylated urethanes, hydrophobically modified nonionic polyols, hydroxypropyl methylcellulose and the like.

Also of use in the compositions is an agent to prevent latent image formation. Some silver salts are light sensitive and discolor upon irradiation of light. However, the degree of light sensitivity may be minimized by several techniques known to those who are skilled in the art. For example, storage of the silver halide particles in a low pH environment will minimize discoloration. In general, pH below 7.0 is desired and more specifically, pH below 4.5 is preferred. Another technique to inhibit discoloration involves adding compounds of elements, such as, iron, iridium, rhuthinium, palladium, osmium, gallium, cobalt, rhodium, and the like, to the silver halide particles. These compounds are known in the photographic art to change the propensity of latent image formation; and thus the discoloration of the silver salt. Additional emulsion dopants are described in Research Disclosure, February 1995, Volume 370, Item 37038, Section XV.B., published by Kenneth Mason Publications, Ltd., Dudley Annex, 12a North Street, Elmsworth, Hampshire PO11 7DQ, England. In any event, discoloration due to latent image formation, while cosmetically undesirable for the antimicrobial composition itself, will typically not have a substantially negative impact on the treated substrate, as the silver halide particles are applied at a relatively low concentration.

While the invention has been primarily described with respect to detection of silver halide salts, selection of appropriate dyes having the desired relative adsorption affinities and/or color change properties to achieve detectable differential color change properties for a variety of silver based compositions relative to a particular substrate will be within the skill of the artisan. As described above, detection of silver metal in various embodiments of the invention may be accomplished by employing a dye having adsorption affinity for a silver salt, wherein the dye solution also in situ converts surface of silver metal to a silver salt. Various oxidizing agents, including environmental oxygen, may be used to convert silver metal to silver ions, and dyes solutions may include soluble halide salts, and in particular bromide or iodide salts, in an amount effective to form silver halide surfaces with adsorption affinity for a selected dye, which may enhance the detectable differential color change in the dye solution contacted substrate or treatment solution.

The present invention is not limited to treatment of, and detection of silver-containing antimicrobial agent on, any particular substrate. Any fiber or textile fabric or yarn may be employed, e.g., including, exhaustively any natural or manufactured fibers, and blends thereof. Examples of natural fibers include, cotton (cellulosic), wool, or other natural hair fibers, for example, mohair and angora. Examples of manufactured fibers include synthetics, such as, polyester, polyolefins such as polyethylene and polypropylene, nylon, acrylic, polyamide, polyether block amide including PEBAX, polycarbonate resins, polyvinylpyrolidinone, polyethylene oxide, and the like, as well as their interpolymers and blends, unsaturated polyesters, alkyds, phenolic polymers, amino plastics, epoxy resins, polyurethanes, polysulfides, polystyrene, or, regenerated materials such as cellulosics. As demonstrated in the examples below, e.g., the presence of silver halide particles deposited on the surface of a variety of substrates may be detected with dye solutions comprising selected dyes having a greater adsorption affinity for silver halide relative to that for the substrates, wherein a detectable different color is obtained when adsorbed to silver halide relative to color when adsorbed to the substrate. The target substrate may further include any number of chemistries or applications prior to, during and/or after the application of the antimicrobial composition including, for example, antistatic control agents, flame retardants, soil resistant agents, wrinkle resistant agents, shrink resistant agents, dyes and colorants, brightening agents, UV stabilizers, lubricants, antimigrants, and the like.

The following examples are intended to demonstrate, but not to limit, the invention.

### EXAMPLE 1

This example shows that DYE-1 can discriminate between sections of a fabric that have been treated with an antimicrobial treatment containing AgCl and those that have not.

Padding Bath A was prepared by mixing (1) a silver chloride/gelatin emulsion in water (silver index ˜1.036 kg/mole silver, gelatin level ˜19.2 g/mole) like that described in Example 1 in US 2006/0068024, (2) an acrylic binder dispersion (RHOPLEX® TS-934HS, Rohm and Haas Co., Philadelphia, Pa., USA) and (3) water in the following percentages:

Padding Bath A

| Component | Wt % |
|---|---|
| AgCl | 0.0076 |
| gelatin | 0.0010 |
| acrylic binder | 0.2500 |
| water | 99.740 |

Two sections of a 10 inch long×5 inch wide sample of white Spun Polyester Fabric (Style #777, Testfabrics Inc., West Pittston, Pa., USA) were masked by applying plastic-coated tape across the full width of both sides of the fabric for a distance of 1 inch beginning at a line approximately 2 inches from one end of the sample, and again for a distance of two inches beginning at a line approximately six inches from the same end. In this way the sample was made to contain two bands, one 1 inch wide and a second 2 inches wide that were inaccessible to the components of any liquid bath in which the fabric was immersed.

This masked fabric sample was immersed in Padding Bath A for approximately 5 seconds, removed from the bath and passed through a pressurized nip/roller system in which the pressure was set to achieve a wet pickup [((weight of fabric after bath)-(weight of dry fabric))/(weight of dry fabric)] of approximately 80%. The sample was then heated to 315 F. for approximately 3 minutes to crosslink the acrylic binder. After this heating step, the tape was removed. Once the tape was removed, there was no obvious visual difference between the masked and unmasked areas of the fabric.

Dyeing Solution 1A was prepared by mixing (1) DYE-1, (2) methyl alcohol, (3) potassium iodide and (4) water in the following amounts:

Dyeing Solution 1A

| Component | amount | |
|---|---|---|
| DYE-1 | 0.00996 | grams |
| potassium iodide | 0.06640 | grams |
| methyl alcohol | 100.0 | mls |
| water | 300.0 | mls |

The fabric sample prepared above was immersed in Dyeing Solution 1A for 1 minute at room temperature (˜23.5 C.). The fabric was then removed from the dyeing solution, rinsed in 1 liter of water at room temperature for 1 minute and then dried. Visual inspection of the dried strip showed that the sections of the fabric that had not been covered by tape during the padding operation had adsorbed DYE-1 and had changed color from white to magenta. The two bands that had been masked during the padding operation did not adsorb the dye and remained white.

### EXAMPLE 2

This example shows that the invention process can quantify the amount of a silver-based antimicrobial applied to a fabric.

Padding Baths B thru E were prepared by mixing the same components used to prepare Padding Bath A in Example 1 except that the percentages shown in Table 1 were used. If the silver chloride level in Bath C is taken as 1×, then the silver chloride in Baths B, D, and E are 0×, 2×, 3×, respectively.

TABLE 1

| | Padding Bath | | | |
|---|---|---|---|---|
| Component | B | C | D | E |
| Silver chloride | 0.00000 | 0.00927 | 0.01854 | 0.02781 |
| Gelatin | 0.00127 | 0.00127 | 0.00127 | 0.00127 |
| Acrylic binder | 0.31250 | 0.31250 | 0.31250 | 0.31250 |
| Water | 99.69 | 99.68 | 99.67 | 99.66 |

Separate samples of undyed (white) Spun Polyester Fabric (Testfabrics Inc., style #777) were immersed in Padding Baths B thru E for approximately 5 seconds (designated Examples 2a, 2b, 2c 2d, respectively), removed from the bath and passed through a pressurized nip/roller system in which the pressure was set to achieve a wet pickup of approximately 80%. The samples were then heated to approximately 350 F. for 20 minutes to crosslink the acrylic binder.

Dyeing Solution 1B was prepared by mixing (1) DYE-1, (2) methyl alcohol, (3) sodium iodide and (4) water in the following amounts:

Dyeing Solution 1B

| Component | amount | |
|---|---|---|
| DYE-1 | 0.00499 | grams |
| sodium iodide | 0.00292 | grams |
| methyl alcohol | 10.0 | mls |
| water to a total volume of | 100.0 | mls |

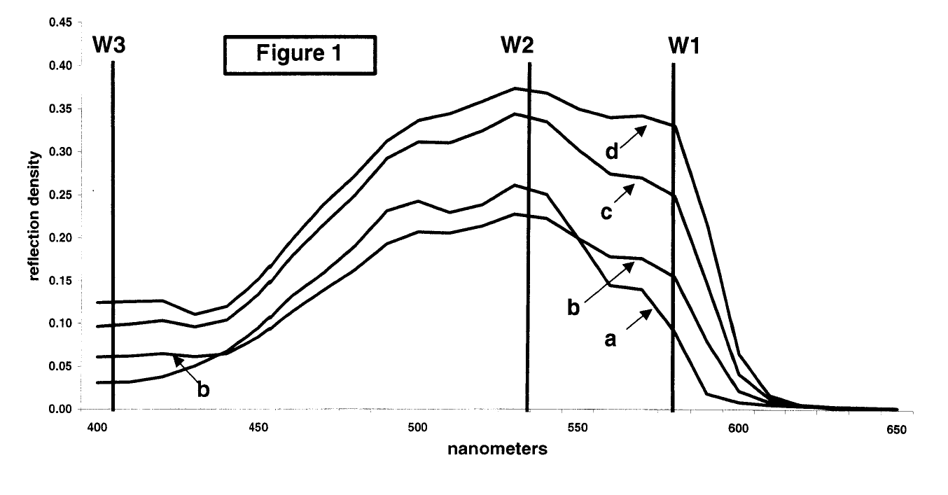

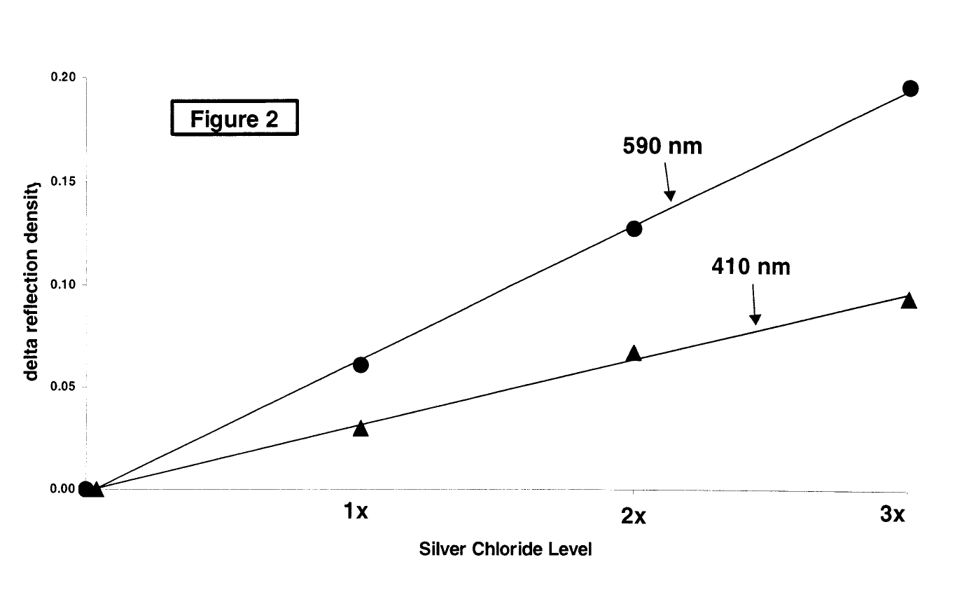

The padded fabric samples Examples 2a thru 2d were dyed using Dyeing Solution 1B according to the following protocol: (1) presoak the samples in room temperature water (˜23 C.) for 1 minute; (2) immerse in Dyeing Solution 1B at 25 C. for 5 minutes; (3) wash in running water for 3 minutes at room temperature; (4) dry. The reflection optical density of the dyed samples was measured as a function of wavelength using a Spectrolino spectrophotometer (GretagMacbeth Corp., Regensdorf, Switzerland), operating in a reflection measurement mode. These reflection optical density spectra are shown in FIG. 1. Curve a in FIG. 1 shows the reflectance spectrum for dyed sample Example 2a, which contained no padded silver chloride. The spectrum of dyed sample Example 2a is characterized by a single, broad band centered at wavelength W2 (˜540 nm). Curves b, c, and d in FIG. 1 are the reflectance spectra for dyed samples of Examples 2b, 2c, and 2d, respectively. They show the growth of a new band—not present in the dyed sample of Example 2a—at wavelength W1 (580-590 nm), that increases in intensity as the level of silver chloride increases. Another band whose intensity also increases with silver chloride level appears in the dyed samples of Examples 2b, 2c, and 2d at wavelength W3 (˜410 nm). Delta Reflection Densities corresponding to the three silver chloride levels used in the dyed samples of Examples 2b, 2c and 2d are shown in Table 2, wherein the Delta Reflection Density represents the increase in refection optical density relative to the reflection optical density of dyed Sample 2a (no padded silver chloride). The Delta Reflection Densities in Table 2 are plotted as a function of padded silver chloride level in FIG. 2, which demonstrates an excellent correlation between the increase in reflection optical density of the dyed samples at wavelengths W1 and W3 and the silver chloride level padded on the dyed fabrics.

TABLE 2

| | | Delta Reflection Density | |
|---|---|---|---|
| | | (Reflection density minus reflection | |
| | Silver Chloride | density of Sample 2a) | |
| | Level in Padding | W3 | W1 |
| Example | Bath | 410 nm | 590 nm |
| 2a | 0x | — | — |
| 2b | 1x | 0.0618 | 0.0789 |
| 2c | 2x | 0.0989 | 0.1458 |
| 2d | 3x | 0.1255 | 0.2147 |

### EXAMPLE 3

This example shows that a number of dyes can be used in the process of the invention to detect a silver-based antimicrobial applied to different types of fabric.

Three fabrics were obtained from Testfabrics Inc.: Spun Polyester (style #777); Cotton Sheeting (style #493); and Polyester/Cotton Blend, 65/35 (style #7436). Samples of each of the fabric types were padded and cured using Padding Baths E and B of Example 2, resulting in sample pairs with and without a silver chloride antimicrobial coating, respectively. The original fabrics were undyed, and showed no color as received or after the padding and curing process.

Dyeing Solutions 2A thru 5 were prepared exactly as Dyeing Solution 1B of Example 2 except that DYE-1 was replaced as shown in Table 3.

TABLE 3

| Dyeing | | |
|---|---|---|
| Solution | Dye | Grams |
| 2A | DYE-2 | 0.00774 |
| 3 | DYE-3 | 0.00774 |
| 4 | DYE-4 | 0.00632 |
| 5 | DYE-5 | 0.00727 |

The six padded fabric samples were dyed using each of the five Dyeing Solutions 1B, 2A, 3, 4, 5 according to the following protocol: (1) presoak the samples in room temperature water (˜23 C.) for 1 minute; (2) immerse in a Dyeing Solution at 60 C. for 1 minute; (3) wash in running water for 3 minutes at room temperature; (4) dry. Reflection optical density spectra were measured for each of the thirty dyed samples produced. The dyed samples are listed in Table 4 along with the wavelengths W2 and W1 (see FIG. 1) for each of the dyes on the fabrics, as well as the reflection optical densities at each of those wavelengths. The color of each of the samples determined from visual inspection of the dyed samples is also given.

TABLE 4

| | | | | | | | | Reflection | |
|---|---|---|---|---|---|---|---|---|---|
| | | | | | | | | density | |
| | | AgCl | | | Reflection | | Reflection | ratio | Sample |
| | | on | | W2 | density | W1 | density | (at W1/at | visual color |
| Sample | Fabric | fabric | Dye | (nm) | at W2 | (nm) | at W1 | W2) | after dyeing |
| 4-A-1 | Polyester | Yes | DYE-1 | 540 | 0.1934 | 580 | 0.2650 | 1.3702 | Magenta |
| 4-A-2 | ″ | No | ″ | ″ | 0.1121 | ″ | 0.0815 | 0.7270 | no color |
| 4-B-1 | Cotton | Yes | ″ | ″ | 0.5922 | ″ | 0.1853 | 0.3129 | magenta/salmon |
| 4-B-2 | ″ | No | ″ | ″ | 0.5348 | ″ | 0.0969 | 0.1812 | magenta/salmon |
| 4-C-1 | poly/cotton | Yes | ″ | ″ | 0.4659 | ″ | 0.1579 | 0.3389 | Magenta |
| 4-C-2 | ″ | No | ″ | ″ | 0.4672 | ″ | 0.0878 | 0.1879 | Magenta |
| 4-D-1 | polyester | Yes | DYE-2 | 510 | 0.2363 | 540 | 0.2613 | 1.1058 | Magenta |
| 4-D-2 | ″ | No | ″ | ″ | 0.1054 | ″ | 0.0874 | 0.8292 | no color |
| 4-E-1 | cotton | Yes | ″ | ″ | 0.2287 | ″ | 0.1302 | 0.5693 | salmon/magenta |
| 4-E-2 | ″ | No | ″ | ″ | 0.1971 | ″ | 0.0160 | 0.0812 | lime-yellow |
| 4-F-1 | poly/cotton | Yes | ″ | ″ | 0.1479 | ″ | 0.1037 | 0.7011 | Magenta |
| 4-F-2 | ″ | No | ″ | ″ | 0.1354 | ″ | 0.0411 | 0.3035 | no color |
| 4-G-1 | polyester | Yes | DYE-3 | 520 | 0.1544 | 550 | 0.1818 | 1.1775 | Magenta |
| 4-G2 | ″ | No | ″ | ″ | 0.0958 | ″ | 0.0748 | 0.7808 | no color |
| 4-H-1 | cotton | Yes | ″ | ″ | 0.1270 | ″ | 0.0989 | 0.7787 | Salmon |
| 4-H-2 | ″ | No | ″ | ″ | 0.1225 | ″ | 0.0282 | 0.2302 | Lime |
| 4-I-1 | poly/cotton | Yes | ″ | ″ | 0.1048 | ″ | 0.0973 | 0.9284 | Magenta |
| 4-I-2 | ″ | No | ″ | ″ | 0.1016 | ″ | 0.0493 | 0.4852 | no color |
| 4-J-1 | polyester | Yes | DYE-4 | 440 | 0.2100 | 470 | 0.1651 | 0.7862 | lime-yellow |
| 4-J-2 | ″ | No | ″ | ″ | 0.1619 | ″ | 0.0724 | 0.4472 | lime-yellow |
| 4-K-1 | cotton | Yes | ″ | ″ | 0.3767 | ″ | 0.1064 | 0.2825 | lime-yellow |
| 4-K-2 | ″ | No | ″ | ″ | 0.3526 | ″ | 0.0260 | 0.0737 | lime-yellow |
| 4-L-1 | poly/cotton | Yes | ″ | ″ | 0.2519 | ″ | 0.1220 | 0.4843 | lime-yellow |
| 4-L-2 | ″ | No | ″ | ″ | 0.2216 | ″ | 0.0133 | 0.0600 | lime-yellow |
| 4-M-1 | polyester | Yes | DYE-5 | 440 | 0.2669 | 470 | 0.2905 | 1.0884 | Yellow |
| 4-M-2 | ″ | No | ″ | ″ | 0.1447 | ″ | 0.0848 | 0.5860 | no color |
| 4-N-1 | cotton | Yes | ″ | ″ | 0.4378 | ″ | 0.1826 | 0.4171 | yellow-green |
| 4-N-2 | ″ | No | ″ | ″ | 0.4197 | ″ | 0.0456 | 0.1086 | yellow-green |
| 4-O-1 | poly/cotton | Yes | ″ | ″ | 0.2677 | ″ | 0.1760 | 0.6575 | yellow-green |
| 4-O-2 | ″ | No | ″ | ″ | 0.2144 | ″ | 0.0168 | 0.0784 | yellow-green |

The data in Table 4 show for each of the dyes tested the presence of silver chloride on each of the three fabrics correlates with the appearance of a new band at higher wavelength (W1) in the reflectance spectrum such that for each sample pair (with and without padded silver chloride) the ratio of the reflection density at W1 to that at W2 is significantly higher for the sample containing padded silver chloride. For all of the dyes except DYE-4, the presence (or absence) of the silver chloride coating on polyester can be directly assessed visually (color versus no color) without having to measure the reflection spectrum, while for DYE-2 and DYE-3, there is sufficient color change for all three fabrics when silver chloride is present to allow a direct, visual verification that silver chloride had been applied to the fabrics.

### EXAMPLE 4

This example shows the impact of the presence of specific soluble halide ions, particularly iodide ion, in the dyeing solutions used to detect a padded silver chloride antimicrobial using the process of the invention.

Dyeing Solutions 1C thru 1E were prepared as described for Dyeing Solution 1B of Example 2 and Dyeing Solutions 2B thru 2D as Dyeing Solution 2A of Example 3, except that the sodium iodide was either omitted or replaced by an equal molar concentration of the sodium halide shown in Table 5.

TABLE 5

| Dyeing Solution | Dye | Sodium Halide |
|---|---|---|
| 1C | DYE-1 | none |
| 1D | DYE-1 | chloride |
| 1E | DYE-1 | bromide |
| 2B | DYE-2 | none |
| 2C | DYE-2 | chloride |
| 2D | DYE-2 | bromide |

The polyester fabric (Spun Polyester style #777) samples prepared with and without padded silver chloride in Example 3 were dyed using Dyeing Solutions 1B thru 1E and 2A thru 2D according to the protocol described in Example 3 and the reflection optical density spectra of the dyed samples were recorded. The reflection densities for each sample at wavelengths W1 and W2 (see FIG. 1) are listed in Table 6 along with the ratios of the reflection density at W1 to that at W2, along with visual color observations made on the dyed samples.

TABLE 6

| | | | | | | | | | reflection | |
|---|---|---|---|---|---|---|---|---|---|---|
| | | | | | | | | | density | |
| | AgCl | | | Dyeing | | reflection | | reflection | ratio | |
| | on | | Dyeing | Solution | W2 | density | W1 | density | (at W1/at | sample color |
| Sample | fabric | Dye | Solution | halide | (nm) | at W2 | (nm) | at W1 | W2) | after dyeing |
| 6-A-1 | Yes | DYE-1 | 1C | none | 540 | 0.1346 | 580 | 0.1023 | 0.7600 | very slightly pink |
| 6-A-2 | No | ″ | ″ | ″ | ″ | 0.1206 | ″ | 0.0832 | 0.6899 | very slightly pink |
| 6-B-1 | Yes | ″ | 1D | chloride | ″ | 0.1381 | ″ | 0.1047 | 0.7581 | darker pink |
| 6-B-2 | No | ″ | ″ | ″ | ″ | 0.1068 | ″ | 0.0750 | 0.7022 | very slightly pink |
| 6-C-1 | Yes | ″ | 1E | bromide | ″ | 0.1522 | ″ | 0.1472 | 0.9671 | still darker pink |
| 6-C-2 | No | ″ | ″ | ″ | ″ | 0.1184 | ″ | 0.0812 | 0.6858 | very slightly pink |
| 6-D-1 | Yes | ″ | 1B | iodide | ″ | 0.2089 | ″ | 0.2170 | 1.0388 | full magenta |
| 6-D-2 | No | ″ | ″ | ″ | ″ | 0.1115 | ″ | 0.0774 | 0.6942 | very slightly pink |
| 6-E-1 | Yes | DYE-2 | 2B | none | 510 | 0.1109 | 540 | 0.0763 | 0.6880 | no color |
| 6-E-2 | No | ″ | ″ | ″ | ″ | 0.1062 | ″ | 0.0795 | 0.7486 | no color |
| 6-F-1 | Yes | ″ | 2C | chloride | ″ | 0.0996 | ″ | 0.0737 | 0.7400 | no color |
| 6-F-2 | No | ″ | ″ | ″ | ″ | 0.1109 | ″ | 0.0839 | 0.7565 | no color |
| 6-G-1 | Yes | ″ | 2D | bromide | ″ | 0.1243 | ″ | 0.1082 | 0.8705 | very slight orange |
| 6-G-2 | No | ″ | ″ | ″ | ″ | 0.0958 | ″ | 0.0724 | 0.7557 | no color |
| 6-H-1 | Yes | ″ | 2A | iodide | ″ | 0.1941 | ″ | 0.2200 | 1.1334 | full magenta |
| 6-H-2 | No | ″ | ″ | ″ | ″ | 0.1085 | ″ | 0.0800 | 0.7373 | no color |

For DYE-1, the data in Table 6 show that the higher wavelength band at W1 appears in the dyed samples containing padded silver chloride even when halide ion is absent from the dyeing solution. This is evidenced by the observation that the ratio of the reflection density at W1 to that at W2 increases when silver chloride is present on the coated fabric. While the increase is relatively modest in the absence of halide in the dye solution, as well as when chloride is added to the dye solution, the band begins to appear much more prominently when bromide is present in the dyeing solution, and increases dramatically in intensity when iodide is present in the dyeing solution. For DYE-2, the effects of bromide ion and iodide ion are even more dramatic as no significant increase in the reflection density ratio (W1/W2) is seen until either bromide or iodide is present in the dyeing solution. These observations are also reflected in the visual appearance of the dyed samples. For DYE-2, e.g., only when iodide was used in the dyeing solutions did the dyed samples show the full magenta color associated with the band at W1.

The invention has been described in detail with particular reference to certain preferred embodiments thereof, but it will be understood that variations and modifications can be effected within the spirit and scope of the invention.

1. A method for testing for presence of silver metal or a silver salt on a surface of substrate, comprising
a) contacting the substrate with a dye solution, wherein the dye solution comprises a dye selected to provide a detectable differential color change in the dye solution contacted substrate for a substrate having silver metal or silver salt on a surface thereof relative to a substrate not having silver metal or silver salt on a surface thereof, and
b) detecting a presence or absence of the differential color change in the dye solution contacted substrate.

2. The method of claim 1, wherein the dye solution comprises a dye selected to have a greater adsorption affinity for a silver salt relative to that for the substrate or to have a detectable different color when adsorbed to a silver salt relative to color when adsorbed to the substrate, and wherein the dye solution tests directly for the presence of a silver salt on a surface of the substrate, or tests indirectly for the presence of silver metal by converting a surface of the silver metal in situ to a silver salt.

3. The method of claim 2, wherein the dye solution comprises a dye having a greater adsorption affinity for silver halide relative to that for the substrate or has a detectable different color when adsorbed to silver halide relative to color when adsorbed to the substrate, and wherein the dye solution further comprises soluble halide salt and the method tests for the presence of metallic silver by in situ conversion of the surface of the metallic silver to silver halide in the presence of environmental oxygen or added oxidizing agent dissolved in the dye solution.

4. The method of claim 1, wherein the differential color change is human visually detectable.

5. The method of claim 1, wherein the dye is selected based on a color of the substrate to provide a human visually detectable differential color change.

6. The method of claim 1, wherein the method is performed on a test substrate previously treated with a substrate processing solution to verify presence or absence of silver metal or silver salt in the substrate processing solution.

7. The method of claim 1, wherein the method tests for presence of silver halide particles deposited on a surface of a substrate, and the dye solution comprises a dye having a greater adsorption affinity for silver halide relative to that for the substrate or has a detectable different color when adsorbed to silver halide relative to color when adsorbed to the substrate.

8. The method of claim 7, wherein the dye comprises a photographic sensitization dye.

9. The method of claim 7, wherein the dye comprises a cyanine dye including two basic heterocyclic nuclei joined by a methine linkage.

10. The method of claim 9, wherein the dye comprises a quinolinium, benzoxazolium, or benzothiazolium dye.

11. The method of claim 9, wherein the dye comprises a salt of 1-ethyl-2-((1-ethyl-2(1H)-quinolinylidene)methyl)-quinolinium.

12. The method of claim 7, wherein the silver halide particles are predominantly silver chloride.

13. The method of claim 12, wherein the dye solution further comprises soluble bromide or iodide ions in an amount effective to enhance the detectable differential color change in the dye solution contacted substrate obtained for a substrate having silver chloride particles on a surface thereof relative to a substrate not having silver chloride particles on a surface thereof.

14. A method of coating a substrate with a silver-containing composition to provide antimicrobial properties comprising:
providing a composition comprising a silver-containing antimicrobial agent;
providing a substrate;
coating the substrate with said composition; and
verifying presence of silver-containing antimicrobial agent deposited on a surface of the substrate by contacting the substrate with a dye solution, wherein the dye solution comprises a dye selected to provide a detectable differential color change in the dye solution contacted substrate for a substrate having silver-containing antimicrobial agent on a surface thereof relative to a substrate not having silver-containing antimicrobial agent on a surface thereof, and detecting a presence or absence of the differential color change in the dye solution contacted substrate.

15. The method of claim 14, wherein the composition comprises water, silver halide particles, and a binder; and wherein the dye solution comprises a dye having a greater adsorption affinity for silver halide relative to that for the substrate or has a detectable different color when adsorbed to silver halide relative to color when adsorbed to the substrate.

16. The method of claim 15, wherein the silver halide particles are predominantly silver chloride, and wherein the dye solution further comprises soluble bromide or iodide ions in an amount effective to enhance the detectable differential color change in the dye solution contacted substrate obtained for a substrate having silver chloride particles on a surface thereof relative to a substrate not having silver chloride particles on a surface thereof.

17. A method for testing for presence of silver-containing antimicrobial agent in a treatment solution, comprising
a) contacting a sample of the treatment solution with a dye solution, wherein the dye solution comprises a dye selected to provide a detectable differential color change in the dye solution contacted treatment solution for a treatment solution having silver-containing antimicrobial agent therein relative to a treatment solution not having silver-containing antimicrobial agent therein, and
b) detecting a presence or absence of the differential color change in the dye solution contacted treatment solution.

18. The method of claim 17, further comprising treating a substrate with the treatment solution to coat the substrate with the silver-containing antimicrobial agent.

19. The method of claim 17, wherein the treatment solution comprises water, silver halide particles, and a binder; and wherein the dye solution comprises a dye having adsorption affinity for silver halide and which provides a detectable different color when adsorbed to silver halide relative to color when not adsorbed to silver halide.

20. The method of claim 19, wherein the silver halide particles are predominantly silver chloride, and wherein the dye solution further comprises soluble bromide or iodide ions in an amount effective to enhance the detectable differential color change in the dye solution contacted treatment solution for a treatment solution having silver chloride particles therein relative to a treatment solution not having silver chloride particles therein.
